(19) United States
(12) Patent Application Publication
(10) Pub. No.: US 2022/0023545 A1
(43) Pub. Date: Jan. 27, 2022

(54) PRECISION SYRINGE PLUNGER WITH SEPARATELY MANUFACTURED AND IRREVERSIBLY COUPLED PLUNGER ARM AND PLUNGER ROD

(71) Applicant: Board of Regents of the University of Nebraska, Lincoln, NE (US)

(72) Inventors: Donny Suh, Omaha, NE (US); Tyler Scherr, Omaha, NE (US); Ronald Linke, Elkhorn, NE (US); Samantha Busch, Indian Land, SC (US); Phillip M. Leopold, Windermere, FL (US); Christopher Steadham, Harrisburg, NC (US); Brian Walsh, Charlotte, NC (US)

(21) Appl. No.: 17/496,344

(22) Filed: Oct. 7, 2021

Related U.S. Application Data
(63) Continuation of application No. PCT/US2021/039054, filed on Jun. 25, 2021.
Application No. PCT/US2021/039054 is a continuation-in-part of application No. 16/484,171, filed on Aug. 7, 2019, now Pat. No. 11,167,091.
(60) Provisional application No. 63/044,554, filed on Jun. 26, 2020.
(60) Provisional application No. 62/459,143, filed on Feb. 15, 2017.

(51) Int. Cl. A61M 5/315; A61M 5/31; A61B 5/15

(52) U.S. Cl. CPC A61M 5/31515; A61M 5/31505; A61M 5/3137; A61M 2207/00; A61B 5/150236; A61B 5/150244; A61B 5/150259

(57) ABSTRACT

A precision syringe plunger includes a plunger rod and a separately manufactured plunger arm. The plunger rod includes a base formed at a proximal end of the plunger rod and a first interference fit connector formed below the base. The plunger rod is configured to extend longitudinally into a syringe barrel. The plunger arm includes a second interference fit connector formed at a proximal end of the plunger arm. The second interference fit connector is configured to be irreversibly coupled to the first interference fit connector so that the plunger arm extends longitudinally along an outer surface of the syringe barrel. The plunger arm further includes a distally located projection. The projection is configured to receive an applied force that causes an entirety of the syringe plunger to move relative to the syringe barrel so that the plunger rod is actuated through a portion of the syringe barrel.

### CROSS-REFERENCE TO RELATED APPLICATIONS

The present application is a Continuation of International Application No. PCT/US2021/039054, filed Jun. 25, 2021, and titled “PRECISION SYRINGE PLUNGER WITH SEPARATELY MANUFACTURED AND IRREVERSIBLY COUPLED PLUNGER ARM AND PLUNGER ROD,” which claims the benefit of U.S. Provisional Application No. 63/044,554, filed Jun. 26, 2020, and titled “PRECISION SYRINGE PLUNGER WITH SEPARATELY MANUFACTURED AND IRREVERSIBLY COUPLED PLUNGER ARM AND PLUNGER ROD,” all of which are incorporated herein by reference. The present application is also a Continuation-in-Part of U.S. Nonprovisional application Ser. No. 16/484,171, filed Aug. 7, 2019, and titled “Precision Syringe,” which is a 371 U.S. National Phase of International Application No. PCT/US2018/018383, filed Feb. 15, 2018, and titled “Precision Syringe,” which claims the benefit of U.S. Provisional Application No. 62/459,143, filed Feb. 15, 2017, and titled “Suh Precision Injection Syringe,” all of which are incorporated herein by reference.

### TECHNICAL FIELD

The present invention generally relates to syringes and, more particularly, to syringes for applications requiring high precision.

### BACKGROUND

Syringes are often used to dispense or collect fluids. For example, syringes may be used to dispense and/or inject medications, to collect biological fluid samples, and so forth. It is often necessary to position a syringe at a specific location and/or in a specific orientation while dispensing or collecting fluid. In some situations, properly positioning the syringe can make it difficult for a user to dispense or collect fluid with the syringe in an accurate and precise manner.

### SUMMARY

A precision syringe is disclosed with a syringe plunger that can be actuated (e.g., by pushing or pulling upon an external portion of the syringe plunger) from a proximal end near a base of the syringe and also from a distal end near a tip of the syringe. In embodiments, the syringe plunger includes an internal portion (a plunger rod) and an external portion (a plunger arm). The plunger rod includes a base formed at a proximal end of the plunger rod. The plunger rod further includes a first interference fit connector formed below the base at the proximal end of the plunger rod. The plunger rod is configured to extend longitudinally from the base into a syringe barrel. The plunger arm includes a second interference fit connector formed at a proximal end of the plunger arm. The second interference fit connector is configured to be coupled to the first interference fit connector so that the plunger arm extends longitudinally from the base along an outer surface of the syringe barrel. The plunger arm further includes a projection formed at a distal end of the plunger arm. The projection is configured to receive an applied force (e.g., a push/pull from a user's finger) that causes an entirety of the syringe plunger to move relative to the syringe barrel so that the plunger rod is actuated through a portion of the syringe barrel (e.g., to dispense fluid from or suction fluid into the syringe barrel).

The plunger rod and the plunger arm may be separately manufactured and then irreversibly coupled by forcing the first and second interference fit connectors together. For example, in embodiments, a method of manufacturing the syringe plunger includes the steps of: (i) molding or printing the plunger rod; (ii) molding or printing the plunger arm; and (iii) irreversibly coupling the plunger rod to the plunger arm by forcing the first and second interference fit connectors together. This manufacturing methodology may provide production line and cost efficiency without comprising structural integrity of the syringe plunger. In particular, it is more resource efficient (and possibly faster) to separately manufacture and then irreversibly couple the plunger rod and plunger arm to produce the syringe plunger when the plunger rod and plunger arm are both manufactured by injection molding.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 is a perspective view of a syringe with a precision syringe plunger, in accordance with an example embodiment of the present disclosure.

FIG. 2 is a cross-sectional side view of the syringe with the precision syringe plunger illustrated in FIG. 1, in accordance with an example embodiment of the present disclosure.

FIG. 3 is a perspective front view of the precision syringe plunger illustrated in FIG. 1, in accordance with an example embodiment of the present disclosure.

FIG. 4 is a perspective rear view of the precision syringe plunger illustrated in FIG. 1, in accordance with an example embodiment of the present disclosure.

FIG. 5 is a front view of the precision syringe plunger illustrated in FIG. 1, in accordance with an example embodiment of the present disclosure.

FIG. 6 is a rear view of the precision syringe plunger illustrated in FIG. 1, in accordance with an example embodiment of the present disclosure.

FIG. 7 is a side view of the precision syringe plunger illustrated in FIG. 1, in accordance with an example embodiment of the present disclosure.

FIG. 8 is a proximal end view of the precision syringe plunger illustrated in FIG. 1, in accordance with an example embodiment of the present disclosure.

FIG. 9 is a distal end view of the precision syringe plunger illustrated in FIG. 1, in accordance with an example embodiment of the present disclosure.

FIG. 10 is a side view of the syringe illustrated in FIG. 1, in accordance with an example embodiment of the present disclosure.

FIG. 11 is a proximal end view of the syringe illustrated in FIG. 1, in accordance with an example embodiment of the present disclosure.

FIG. 12 is an environmental perspective front view of a precision syringe plunger, in accordance with an example embodiment of the present disclosure.

FIG. 13 is an environmental perspective rear view of the precision syringe plunger illustrated in FIG. 12, in accordance with an example embodiment of the present disclosure.

FIG. 14 is a perspective front view of the precision syringe plunger illustrated in FIG. 12, in accordance with an example embodiment of the present disclosure.

FIG. 15 is a perspective rear view of the precision syringe plunger illustrated in FIG. 12, in accordance with an example embodiment of the present disclosure.

FIG. 16 is a perspective front view of a plunger arm of the precision syringe plunger illustrated in FIG. 12, in accordance with an example embodiment of the present disclosure.

FIG. 17 is a perspective rear view of the plunger arm of the precision syringe plunger illustrated in FIG. 12, in accordance with an example embodiment of the present disclosure.

FIG. 18 is a perspective front view a plunger rod of the precision syringe plunger illustrated in FIG. 12, in accordance with an example embodiment of the present disclosure.

FIG. 19 is a perspective rear view the plunger rod of the precision syringe plunger illustrated in FIG. 12, in accordance with an example embodiment of the present disclosure.

FIG. 20 is a front view of the plunger rod of the precision syringe plunger illustrated in FIG. 12, in accordance with an example embodiment of the present disclosure.

FIG. 21 is a rear view of the plunger arm of the precision syringe plunger illustrated in FIG. 12, in accordance with an example embodiment of the present disclosure.

FIG. 22 is a flow diagram for a method of manufacturing a precision syringe plunger, such as the precision syringe plunger illustrated in FIG. 12, in accordance with an example embodiment of the present disclosure.

### DETAILED DESCRIPTION

Syringes are often employed in delicate procedures where it may be necessary to position or orient a syringe in a manner that makes it difficult to apply a force to (e.g., push or pull upon) the syringe's plunger in order to dispense or collect fluid. To provide a user with ability to actuate a syringe plunger from a position at or near a distal end (e.g., tip) or proximal end (e.g., base) of the syringe, a precision syringe is disclosed with a plunger that can be actuated from the proximal end (base) of the plunger or by alternatively applying force to a distal end of an external portion of the plunger that is near the tip (e.g., dispensing/collecting end) of the syringe. Consequently, the syringe can be placed in a variety of positions and orientations without making it difficult for the plunger to be actuated. For example, the tip of the syringe can be held near a target location while the plunger is actuated to dispense or collect fluid by pushing or pulling a finger-actuated projection at the distal end of the external portion of the plunger that is near the tip of the syringe.

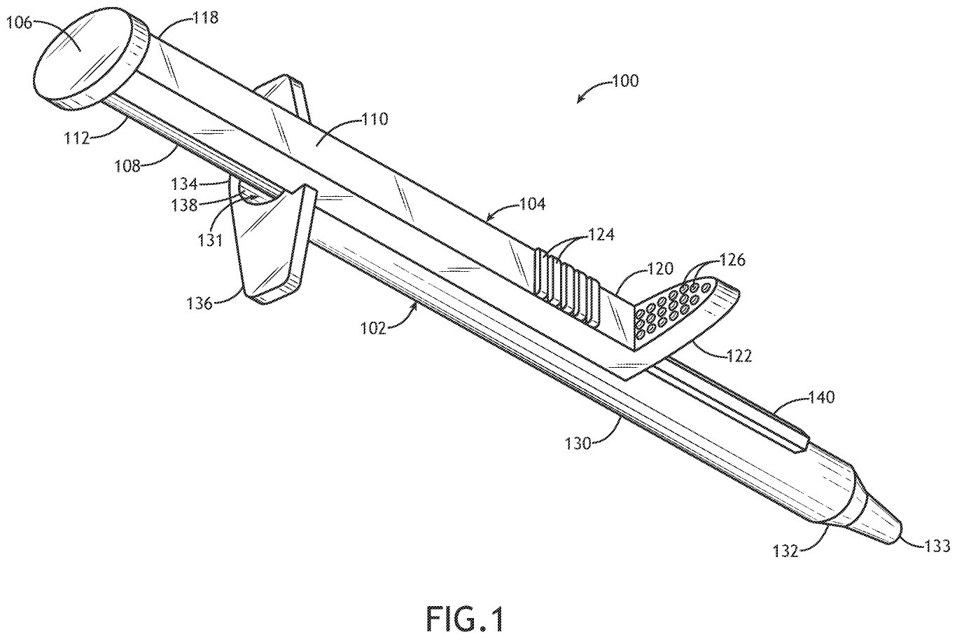

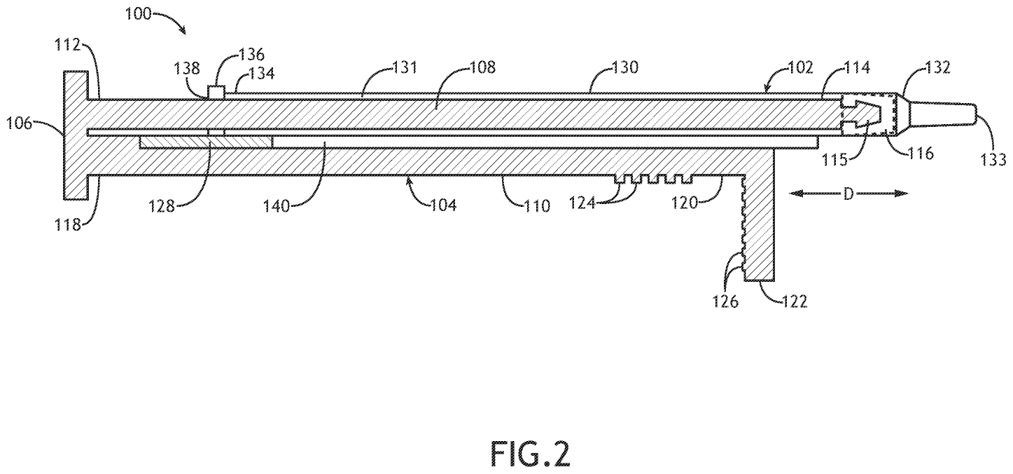

FIGS. 1 and 2 show a precision syringe 100 in accordance with an embodiment of the present disclosure. The syringe 100 includes a syringe barrel 102 and a precision syringe plunger 104 that is configured to be partially disposed within the syringe barrel 102. The syringe barrel 102 includes a body 130 (e.g., a tubular body) that defines a cavity 131 configured to contain fluid (e.g., liquid and/or gas). Examples of fluids that can be collected and/or dispensed by the syringe 100 include, but are not limited to, sample fluids (e.g., biological or other fluid samples), solutions including medications, therapeutic agents, contrast agents, antibiotic/antiseptic solutions, and the like.

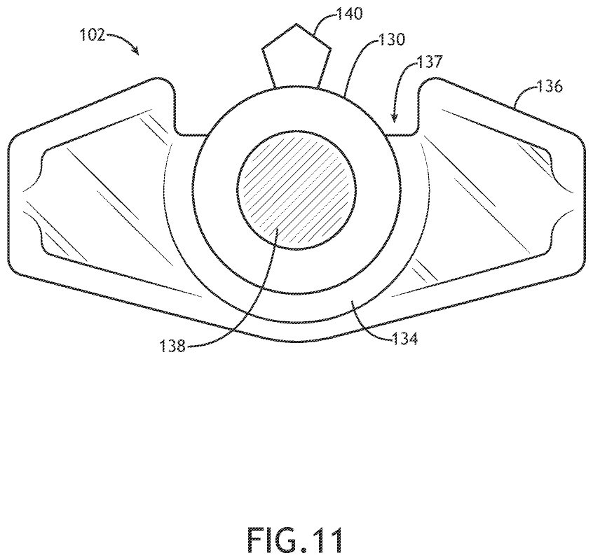

The syringe barrel 102 has a proximal end 134 with an opening 138 that leads into the interior cavity 131 defined by the body 130. The opening 138 is configured to receive an internal portion of the syringe plunger 104, referred to herein as the “plunger rod 108.” The plunger rod 108 is configured to extend longitudinally into the syringe barrel 102 (i.e., into the cavity 131 defined by the body 130) when the syringe plunger 104 is actuated towards a distal end 132 of the syringe barrel 102. As shown in FIG. 11, the syringe barrel 102 may also have a flange 136 formed around the opening 138. The flange 136 may include a notch 137 that at least partially surrounds an external portion of the syringe plunger 104, referred to herein as the “plunger arm 110,” when the plunger rod 108 is at least partially inserted into the syringe barrel 102. The notch 137 may be opposite a surface of the syringe barrel 102 having indicator lines (e.g., volumetric numbering lines) that are formed and/or printed along the body 130 of the syringe barrel 102.

In embodiments, the distal end 132 of the syringe barrel 102 has a tip 133 formed at the distal end 132 for dispensing and/or collecting fluid. The tip 133 may be tapered or cylindrical. In some embodiments, the tip 133 can include or can be coupled to a needle that is used to penetrate a membrane (e.g., skin or seal of a container) to inject or collect fluid through the membrane. In other embodiments, the tip 133 can be coupled to a tube or nozzle to dispense fluid through the tube or with greater precision or at higher pressure using the nozzle.

In some embodiments, the syringe barrel 102 is at least partially patterned with raised ridges around the circumference of the body 130 except for a portion having indicator lines (e.g., volumetric numbering lines) that are formed and/or printed along the body 130 of the syringe barrel 102. For example, the raised edges may run from the tip 133 to a distance of approximately 10 to 30 mm (e.g., 20 mm) along the outer surface of the syringe barrel 102.

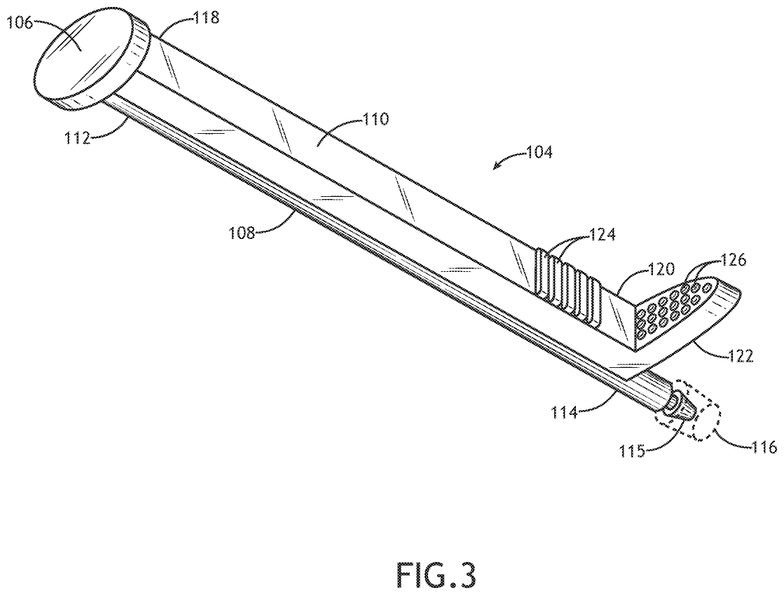

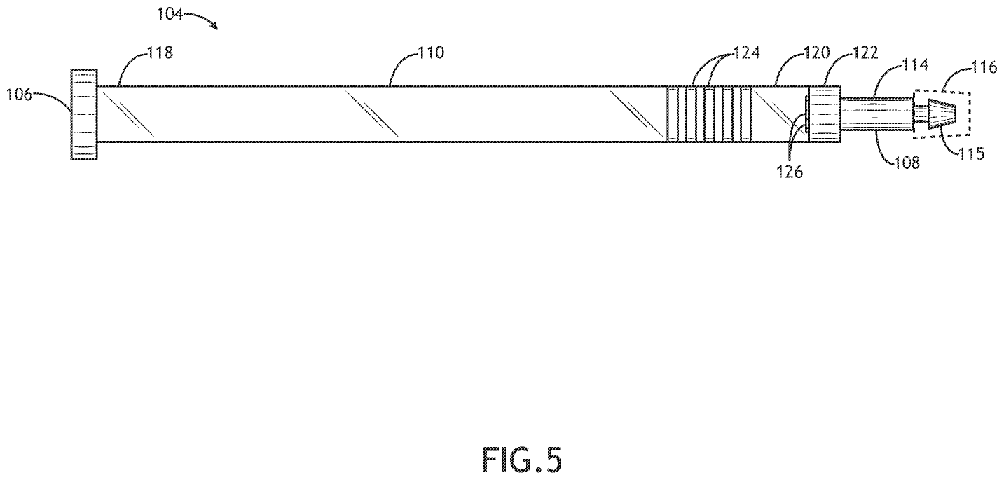

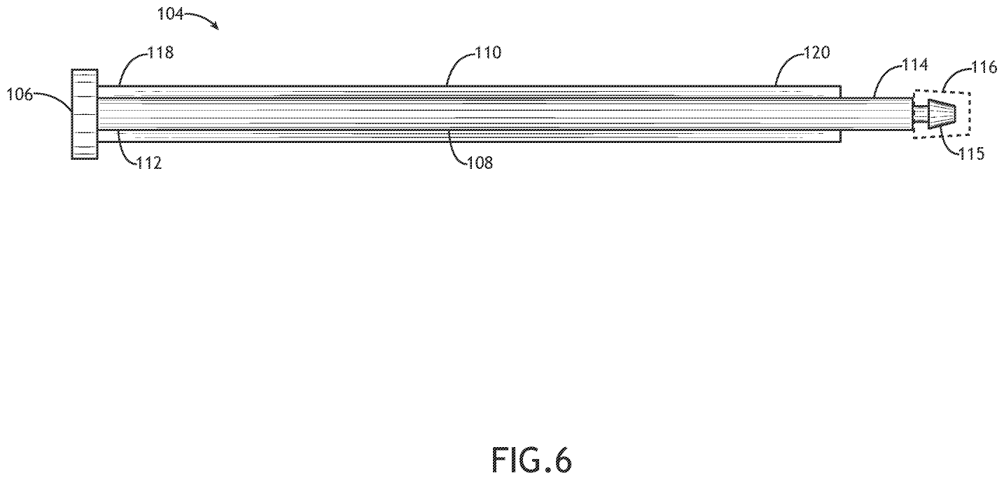

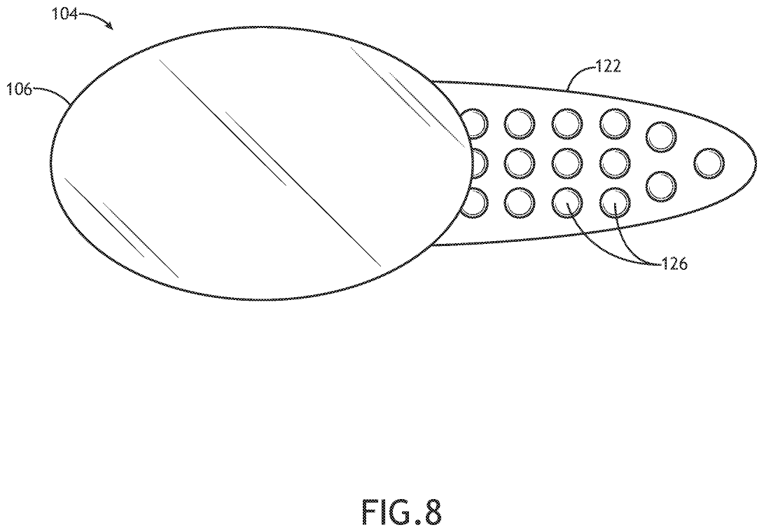

The syringe plunger 104 is shown partially inserted into the syringe barrel in FIGS. 1 and 2. Detailed views of the syringe plunger 104 are also shown FIG. 3 through 9. The syringe plunger 104 includes a base 106 with the plunger rod 108 and plunger arm 110 extending therefrom. The plunger rod 108 extends longitudinally into the syringe barrel 102 and has the base 106 formed at a proximal end 112 of the plunger rod 108. The plunger rod 108 also has a distal end 114 opposite the proximal end 112. The distal end 114 is configured to push fluid out of and/or suction fluid into the syringe barrel 102. In some embodiments, the distal end 114 flares out to contact or nearly contact an inner surface of the body 130 of the syringe barrel 102 so that the distal end 114 can push fluid out of or suction fluid into the syringe barrel 102 (e.g., into or out of the interior cavity 131 defined by the body 130) when the distal end 114 is actuated through a portion of the syringe barrel 102. In some embodiments, the plunger rod 108 has a pusher 116 disposed at the distal end 114 for pushing fluid out of or suctioning fluid into the syringe barrel 102. For example, the pusher 116 can be a plug (e.g., rubber plug, plastic plug, metal plug, ceramic plug, or the like) disposed at the distal end 114 of the plunger rod 108. In some embodiments, the pusher 116 is coupled to the distal end 114 of the syringe plunger 104. For example, the pusher 116 may be coupled to the distal end 114 by an adhesive or by a form-fitting tip 115 that can be forced into the pusher 116 (e.g., as shown in FIG. 2). In other embodiments, the pusher 116 is a flared portion of the distal end 114. For example, the pusher 116 may be made from the same material as the plunger rod 108 and/or part of the same print or mold.

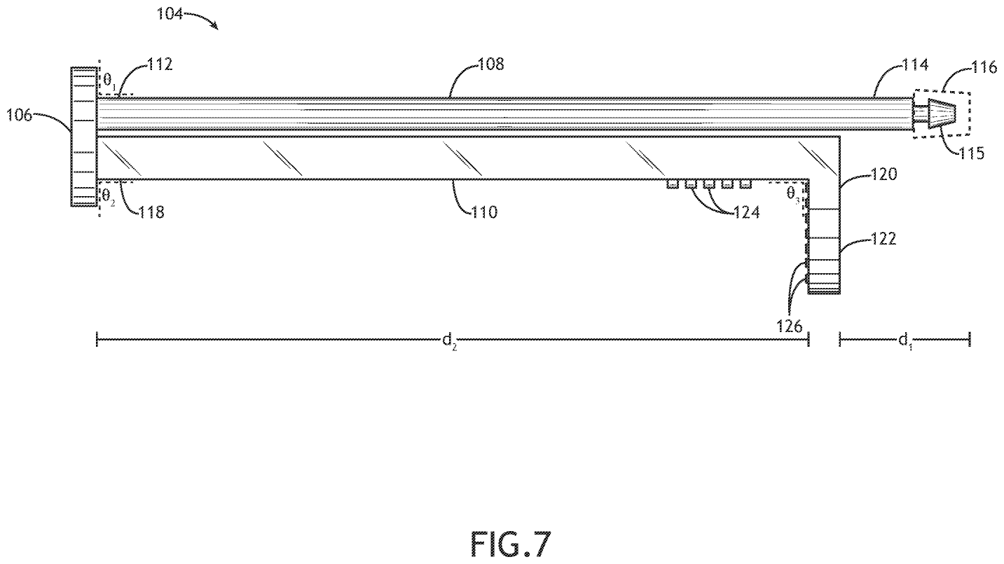

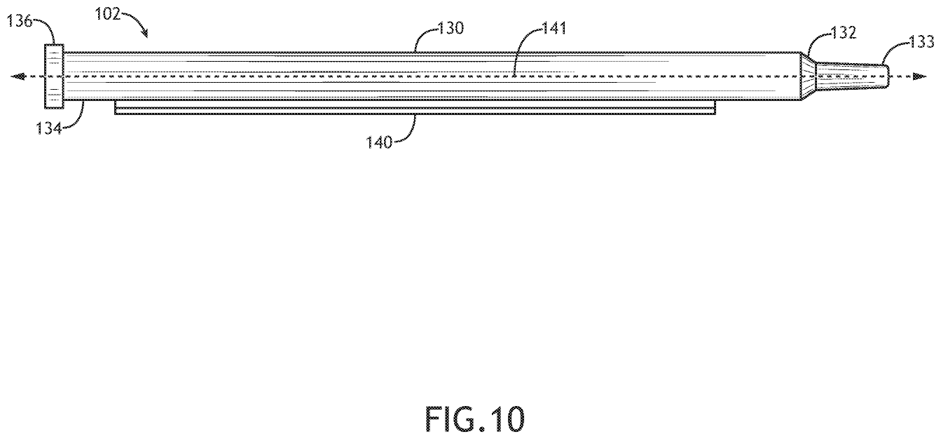

The plunger arm 110 extends longitudinally along an outer surface of the syringe barrel 102 and also has a proximal end 118 coupled to the base 106. The base 106 can be configured to support the plunger rod 108 parallel (or substantially parallel) to the plunger arm 110. For example, the plunger rod 108 and plunger arm 110 may extend from a surface of the base 106 that is perpendicular (or substantially perpendicular) to a longitudinal axis 141 (shown in FIG. 10) of the syringe barrel 102, where each of the plunger rod 108 and plunger arm 110 extend along or parallel to the longitudinal axis 141. In an embodiment shown in FIG. 7, an angle θ₈₁ between the plunger rod 108 and the base 106 is in the range of 80 to 100 degrees (e.g., θ₁ can be a 90 degree angle or nearly 90 degree angle), and an angle θ₂ between the plunger arm 110 and the base 106 is also in the range of 80 to 100 degrees (e.g., θ₂ can be a 90 degree angle or nearly 90 degree angle).

In some embodiments, the plunger arm 110 is connected to the base 106 at a connection site that is approximately 1 to 3 mm from the center of the base 106 (e.g., 1.67 mm out from the center of the base 106). The plunger arm 110 may extend parallel to the longitudinal axis 141 of the syringe barrel 102 to a distance of approximately 10 to 25 mm (e.g., 15 mm) when the plunger rod 108 is fully inserted into the syringe barrel 102. The plunger arm 110 may have a width no greater than a width of the syringe barrel 102 (e.g., the width or diameter of the body 130) and a depth of approximately 1 to 5 mm (e.g., 3 mm).

The syringe plunger 104 can be actuated when force is applied to the base 106. For example, the syringe plunger 104 can move relative to the syringe barrel 102 when the base 106 is pressed, pushed, pulled, or otherwise actuated. The syringe plunger 104 can also be actuated when force is applied to the plunger arm 110. For example, the proximal end 118 of the plunger arm 110 is coupled to the base 106 so that when a distal end 120 of the plunger arm 110 receives an applied force (e.g., a push or pull), the entire plunger 104 moves relative to the syringe barrel 102 so that the plunger rod 108 is actuated through a portion of the syringe barrel 102 (e.g., through the interior cavity 131 defined by the body 130 to dispense fluid from or suction fluid into the interior cavity 131).

In embodiments, the plunger arm 110 has a projection 122 disposed at the distal end 120 of the plunger arm 110. The projection 122 can be configured to receive the applied force (or at least a portion of the applied force). For example, the projection 122 can make it easier to push or pull the distal end 120 of the plunger arm 110 (e.g., with an index finger or other finger of the user) so that the syringe plunger 104 moves relative to the syringe barrel 102. In some embodiments, the projection 122 is perpendicular (or substantially perpendicular) to the longitudinal axis 141 of the syringe barrel 102. For example, as shown in FIG. 7, the projection 122 may extend from the plunger arm 110 at an angle θ₃ in the range of 80 to 100 degrees (e.g., at a 90 degree angle or nearly 90 degree angle). In embodiments where the projection 122 is perpendicular (or substantially perpendicular) to the longitudinal axis 141 of the syringe barrel 102, the projection 122 may be configured to indicate an amount of collected or dispensed fluid. For example, the projection 122 can be aligned with indicator lines (e.g., volumetric numbering lines) that are formed and/or printed along the body 130 of the syringe barrel 102.

In some embodiments, a distance d₁ between the pusher 116 and the projection 122 is less than a distance d₂ between the base 106 and the projection 122. In this regard, the projection 122 can be nearer to the tip 133 of the syringe barrel 102 than it is to the base 136 of the syringe barrel 102 when the syringe plunger 104 is actuated to dispense or collect fluid via the tip 133. In some embodiments, the projection 122 can have, but is not limited to, a teardrop shape that protrudes away from the syringe barrel 102 a distance of approximately 5 to 15 mm (e.g., 11.5 mm) and may also have a width no greater than a width of the syringe barrel 102 (e.g., the width or diameter of the body 130) and a depth of approximately 1 to 5 mm (e.g., 3 mm).

In some embodiments, an entirety of the syringe plunger 104 (e.g., the base 106, the plunger rod 108, and the plunger arm 110) may be a common structure. In this regard, an entirety of the syringe plunger 104 may move relative to the syringe barrel 102 when any portion of the syringe plunger 104 is actuated relative to the syringe barrel 102. For example, the syringe plunger 104 can move relative to the syringe barrel 102 when the base 106 is pressed, pushed, pulled, or otherwise actuated, and similarly when the distal end 120 and/or projection 122 is pressed, pushed, pulled, or otherwise actuated. In some embodiments, the base 106, the plunger rod 108, and the plunger arm 110 are all formed from a common mold or print. For example, the syringe plunger 104 can be a common plastic, metal, or ceramic mold or print. In embodiments, the syringe plunger 104 is formed from one or more biocompatible materials (e.g., biocompatible plastic, metal, and/or ceramic material).

In some embodiments, the plunger arm 110 includes surface texturing 126 on a surface of the projection 122 and/or surface texturing 124 on a surface of the plunger arm 110 that is adjacent to the projection 122. This can make it easier to push or pull the distal end 120 of the plunger arm 110 so that the syringe plunger 104 moves relative to the syringe barrel 102. For example, the surface texturing 124 and/or 126 on the plunger arm 110 and/or the projection 122 can provide an easier to grip surface that can be pushed or pulled with a finger more easily than a smooth surface. In some embodiments, the surface texturing 124 and/or 126 includes at least one indentation, protuberance, pattern of indentations, and/or pattern of protuberances on a surface of the plunger arm 110 and/or projection 122. For example, FIG. 1 shows surface texturing 124 including a line pattern of protuberances and surface texturing 126 including a dot pattern of protuberances. In some embodiments, the indentations or protuberances have a depth/height of approximately 0.2 to 1.5 mm (e.g., 0.5 mm) from the surface of the plunger arm 110 and/or the projection 122 on which the indentations or protuberances are formed.

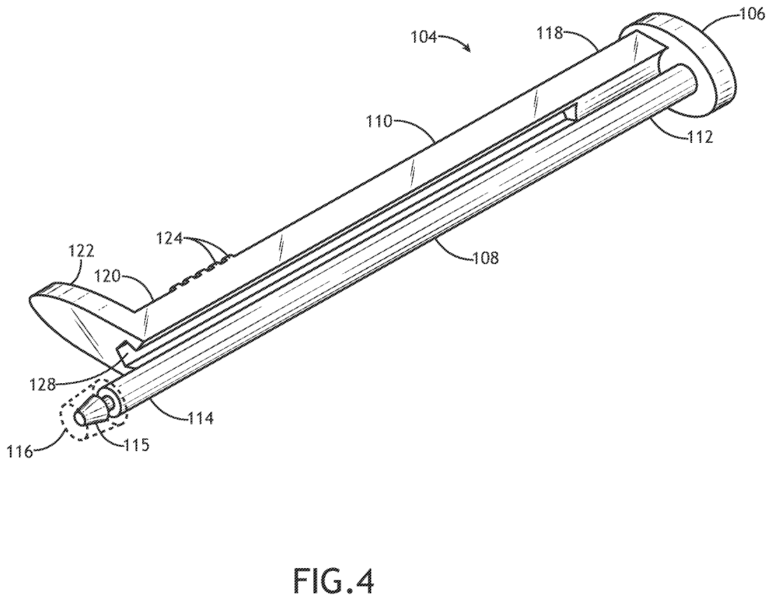

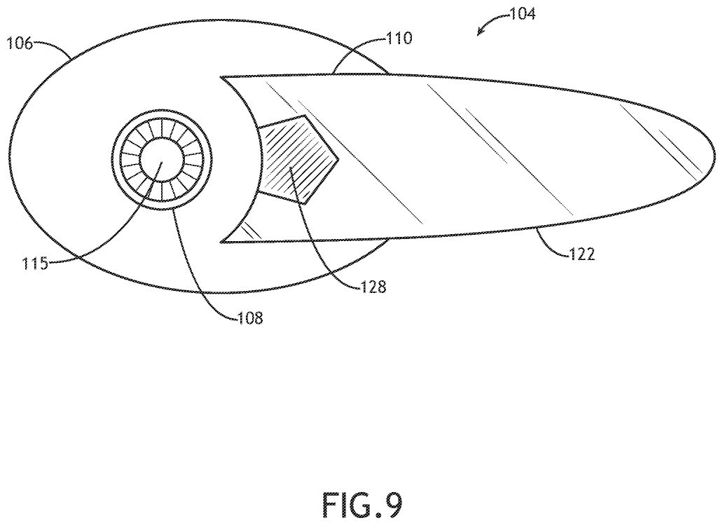

As shown in FIGS. 4 and 9, the plunger arm 110 can also include an indentation 128 that extends longitudinally through at least part of the plunger arm 110. Referring again to FIGS. 1 and 2, the indentation 128 can be configured to fit over at least a portion of a guide 140 on the syringe barrel 102 so that the plunger arm 110 slides along the guide 140 in a longitudinal direction D relative to the syringe barrel 102 when the syringe plunger 104 is actuated (e.g., by pressing, pushing, or pulling upon the base 106, distal end 120, and/or projection 122). For example, FIGS. 10 and 11 show side and end views, respectively, of the syringe barrel 102 with the guide 140 extending longitudinally along the outer surface of the syringe barrel 102 (e.g., along at least a portion of the body 130). In embodiments, the guide 140 is opposite a surface of the syringe barrel 102 having indicator lines (e.g., volumetric numbering lines) that are formed and/or printed along the body 130 of the syringe barrel 102.

As can be seen in FIGS. 9 and 11, the guide 140 may have a shape that is cooperative with a shape of the indentation 128 formed within the plunger arm 110 so that one or more inner surfaces of the indentation 128 are in contact with one or more surfaces of the guide 140 when the syringe plunger 104 (i.e., the plunger rod 108) is at least partially inserted within the syringe barrel 102. The guide 140 can have any shape that is cooperative with the shape of the indentation 128. For example, FIGS. 9 and 11 show the guide 140 and the indentation 128 having pentagonal cross-sections, where the guide 140 has a pentagonal cross-section with one side being curved in accordance with a curvature of the syringe barrel 102 (e.g., curving around at least a portion of the body 130). The guide 140 and the indentation 128 can have any cross-sectional shape, for example, triangular cross-sections, square cross-sections, circular cross-sections, or the like. In any configuration, the guide 140 may have one side or edge that is curved or otherwise shaped so that it forms a continuous (or substantially continuous) interface with the body 130 of the syringe barrel 102.

The guide 140 may be configured to at least partially constrain a movement of the syringe plunger 104 relative to the syringe barrel 102. For example, the guide 140 may be configured to at least partially prevent the plunger arm 110 from moving perpendicular to a longitudinal axis 141 of the syringe barrel 102 (e.g., from moving in any direction other than a longitudinal direction D along the outer surface of the syringe barrel 102). This provides stability for the user when the syringe plunger 104 is being actuated and can help prevent inaccurate dosage or accidental repositioning of the syringe 100.

In an example use scenario, the syringe 100 can be held so that a distal end 132 of the syringe barrel 102 is supported between a user's thumb and middle finger. The syringe plunger 104 can then be actuated by applying force to the distal end 120 of the plunger arm 110 and/or to the projection 122. This allows the user to actuate the syringe plunger 104 with the user's index finger while maintaining the syringe 100 in a selected position and/or orientation with the user's thumb and middle finger. The user is not required to use another hand or readjust his/her grip of the syringe 100.

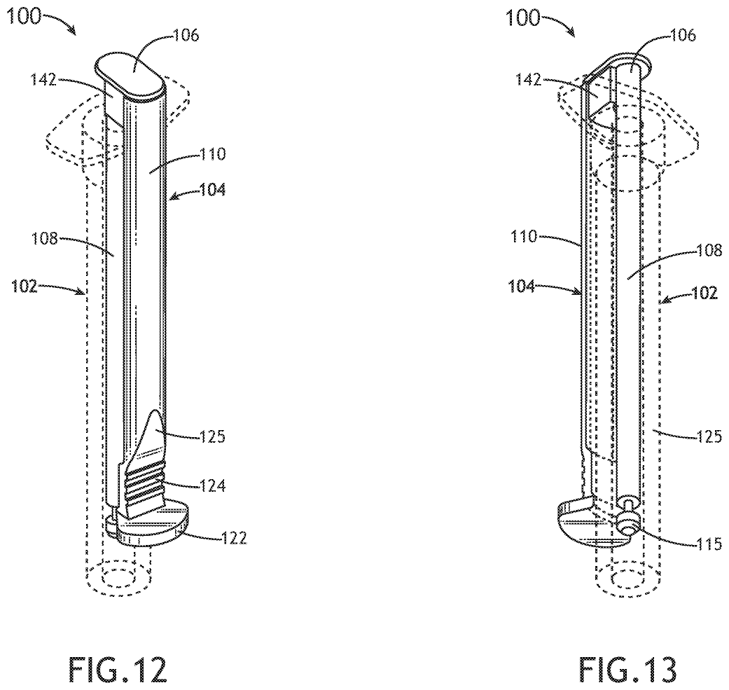

FIGS. 12 through 21 illustrate another embodiment of the precision syringe 100, which is described below. Unless otherwise stated, reference numerals that are the same as the reference numbers used in FIGS. 1 through 11 identify the same components described above with reference to FIGS. 1 through 11. Furthermore, embodiments of any components described with reference to FIGS. 1 through 11 may be interchanged with embodiments of the same components described with reference to FIGS. 12 through 21, and vice versa.

In the embodiment illustrated in FIGS. 12 through 21, the syringe plunger 104 includes separately manufactured and irreversibly coupled internal (plunger rod 108) and external (plunger arm 110) portions. For example, as shown in FIGS. 12 and 13, the plunger rod 108 and the plunger arm 110 may be secured together by an irreversible coupling 142 at the proximal end of the syringe plunger 104. In some embodiments, the irreversible coupling 142 is directly below the base 106 of the syringe plunger 104, such that the base 106 and irreversible coupling 142 form a base structure for the entirety of the syringe plunger 104. The plunger rod 108 is configured to extend longitudinally from the base 106 (and irreversible coupling 142) into the syringe barrel 102, and the plunger arm 110 is configured to extend longitudinally from the base 106 (and irreversible coupling 142) along the outer surface of the syringe barrel 102.

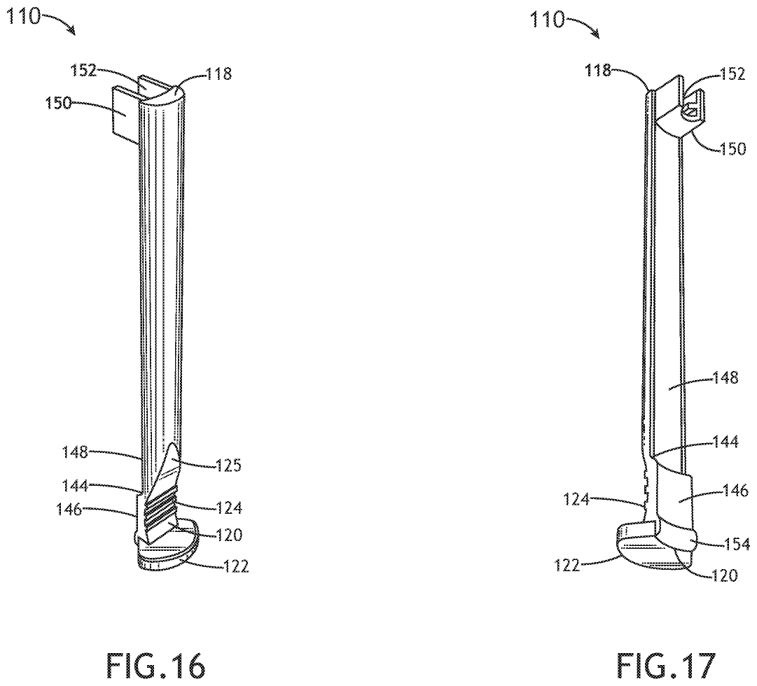

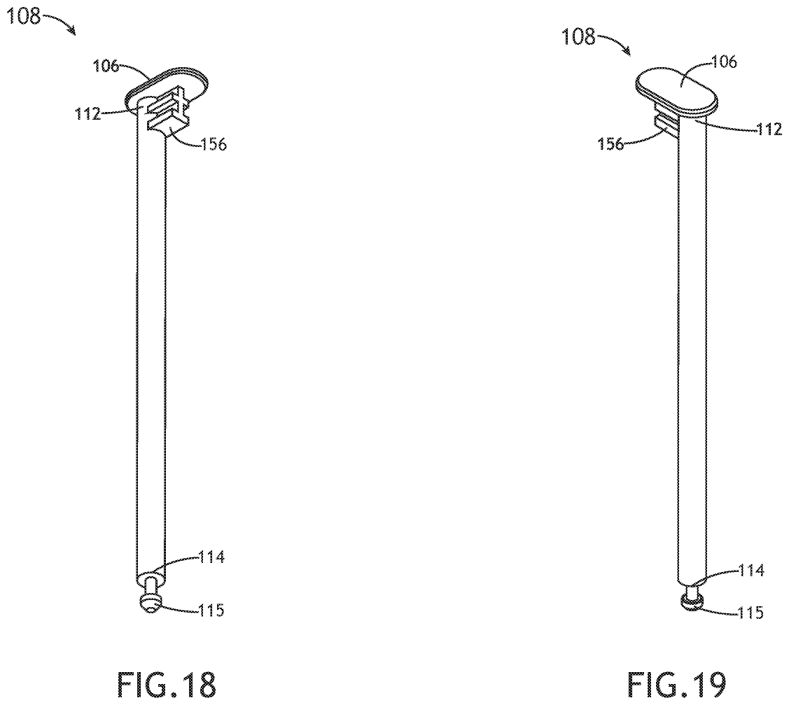

As shown in FIGS. 18 and 19, the plunger rod 108 includes the base 106, which is formed at the proximal end 112 of the plunger rod 108. The plunger rod 108 further includes an interference fit connector 156 formed below the base 106 at the proximal end 112 of the plunger rod 108. As shown in FIGS. 16 and 17, the plunger arm 110 also includes an interference fit connector 150 formed at the proximal end 118 of the plunger arm 110. The interference fit connector 150 of the plunger rod 108 is configured to be coupled to the interference fit connector 156 of the plunger arm 110 to form the irreversible coupling 142.

The plunger arm 110 further includes the projection 122 formed at the distal end 120 of the plunger arm 110. The projection 122 is configured to receive an applied force (e.g., a push/pull from a user's index finger or other finger) that causes the entirety of the syringe plunger 104 to move relative to the syringe barrel 102 so that the plunger rod 108 is actuated through a portion of the syringe barrel 102 (e.g., to dispense fluid from or suction fluid into the syringe barrel 102).

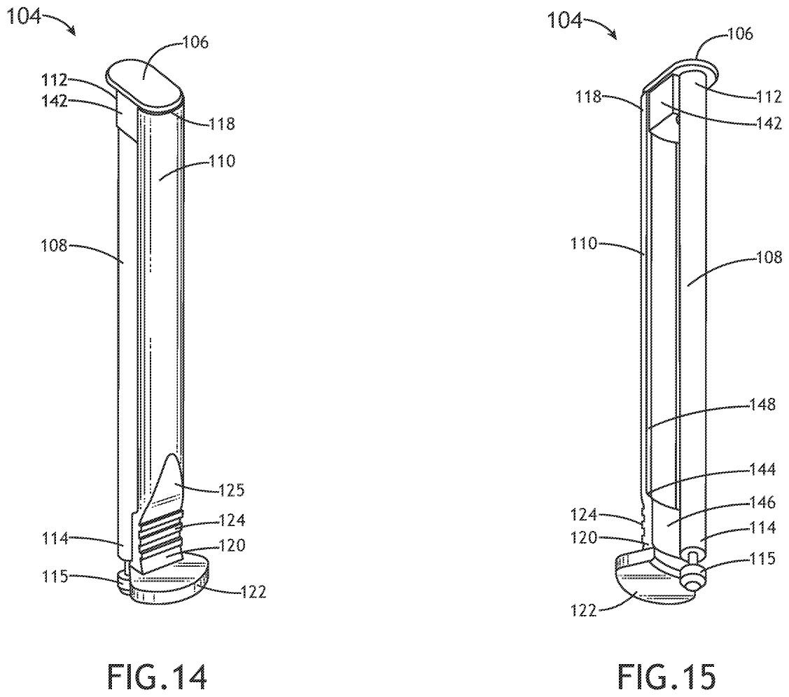

As shown in FIGS. 12 and 13, the projection 122 may be a circular sector or segment configured to be pushed by an index finger (or other finger) of a user to cause the entirety of the syringe plunger 104 to move relative to the syringe barrel 102. In some embodiments, the projection 122 is a circular sector or segment with an arc measure in the range of 90 to 180 degrees (e.g., approximately 120 degrees). As shown in FIGS. 14 and 15, the projection 122 may protrude and flare out from the distal end 120 of the plunger arm 110. Furthermore, the projection 122 may be substantially perpendicular to the plunger rod 108 and to the remainder of the plunger arm 110.

In some embodiments, the plunger arm 110 includes an indented portion 125 near the distal end 120 of the plunger arm 110. For example, in FIG. 14, the plunger arm 104 includes an inwardly curved region (indented portion 125) directly above the projection 122. The indented portion 125 of the plunger arm 104 may have a curvature configured to conform to the index finger (or other finger) of the user for improved grip. The plunger arm 110 may further include surface texturing 124 (e.g., one or more indentations, protuberances, pattern of indentations, and/or pattern of protuberances) near the distal end 120 of the plunger arm 110. For example, in FIG. 14, the indented portion 125 of the plunger arm 110 includes surface texturing 124 to further improve the grip of the index finger (or other finger) of the user used to actuate the syringe plunger 104 by pushing/pulling the distal end 120 (e.g., projection 122 and/or indented portion 125) of the plunger arm 110.

The plunger arm 110 may further include structural features that allow for reduced friction between the plunger arm 110 and the syringe barrel 102 when the entirety of the syringe plunger 104 to moves relative to the syringe barrel 102. For example, in some embodiments, a lower portion 146 of the plunger arm 110 is configured to contact the syringe barrel 102, and an upper portion 148 of the plunger arm 110 is outdented from the lower portion 146 of the plunger arm 110 to prevent or reduce contact between the upper portion 148 of the plunger arm 110 and the syringe barrel 102 when the syringe plunger 104 to moves relative to the syringe barrel 102. In this regard, the plunger arm 110 may include an angled segment 144 at a transition point between the lower portion 146 of the plunger arm 110 and the upper portion 148 of the plunger arm 110. In some embodiments, the lower portion 146 of the plunger arm 110 further includes a smooth bump 154 configured to contact the syringe barrel 102 and configured to prevent or reduce contact between a remainder of the lower portion 146 of the plunger arm 110 and the syringe barrel 102 to further reduce friction between the plunger arm 110 and the syringe barrel 102. For example, in FIG. 17, the smooth bump 154 is directly behind the projection 122 at the distal end 120 of the plunger arm 110.

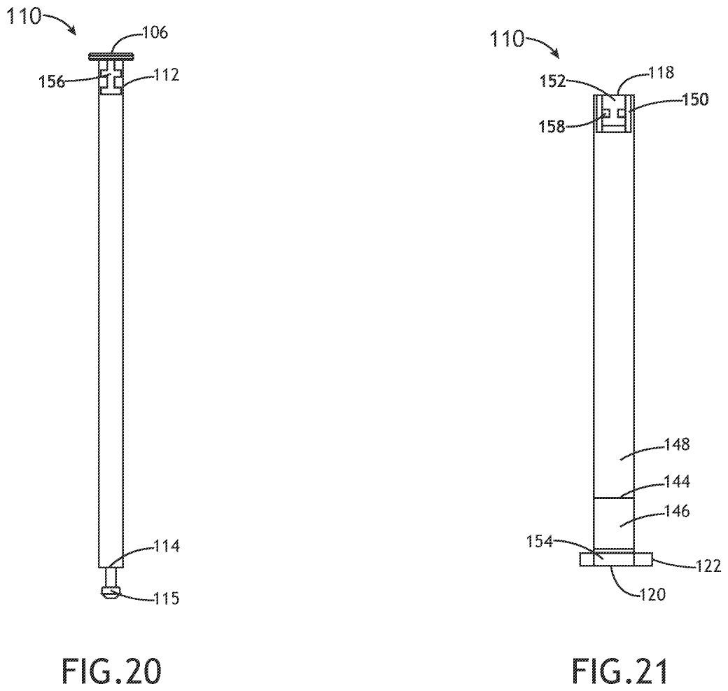

As noted above, the plunger rod 108 and the plunger arm 110 may be separately manufactured and irreversibly coupled. For example, after separately manufacturing the plunger rod 108 and the plunger arm 110 (e.g., by injection molding or 3D printing), the two components may be irreversibly coupled together by forcing together the interference fit connector 156 at the proximal end 112 (or top) of the plunger rod 108 and the interference fit connector 150 at the proximal end 118 (or top) of the plunger arm 110. As shown in FIGS. 20 and 21, the interference fit connectors 156 and 150 may include cooperatively shaped structures configured to mate with one another. In some embodiments, the cooperatively shaped structures couple together irreversibly through the use of features that can only slide relative to one another in a single direction. For example, the shaped structures may include features that can be pressed together but cannot be pulled apart thereafter. In some embodiments, this may be accomplished using projections and notches that click together when the plunger rod 108 and plunger arm 110 are pressed together. In other embodiments, the sizing of the shaped structures causes a forced expansion to occur when the plunger rod 108 and plunger arm 110 are pressed together, such that the components are then stuck together. The irreversible coupling may also be aided by or accomplished through the use of an adhesive between the components or by applying a chemical treatment, heat and/or radiation to fuse the components together.

In embodiments, the interference fit connector 156 of the plunger rod 108 is made of one or more I-shaped extrusions that protrude from the proximal end 112 of the plunger rod 108. For example, in FIGS. 18 through 21, the interference fit connector 156 is an I-shaped extrusion with an additional segment extending vertically therefrom. To accommodate the shape of the interference fit connector 156 of the plunger rod 108, the interference fit connector 150 of the plunger arm 110 may include one or more I-shaped sockets. For example, in FIGS. 16, 17 and 21, the interference fit connector 150 of the plunger arm 110 defines a cavity 152 with internal structures 158 that occupy portions of the cavity 152 so that a cross-section of the unoccupied region of the cavity 152 is I-shaped. In this regard, the interference fit connector 150 of the plunger arm 110 is an I-shaped socket. As shown in FIG. 17, the interference fit connector 150 of the plunger arm 110 (e.g., the I-shaped socket) may be partially curved to conform to a shape of the plunger rod 112, such that the curved portion of the interference fit connector 150 of the plunger arm 110 wraps around a portion of the plunger rod 112.

In the illustrated embodiments, the interference fit connector 156 of the plunger rod 108 is configured to be disposed within the interference fit connector 150 of the plunger arm 110 to form the irreversible coupling 142 directly below the base 106 of the plunger rod 108. However, in other embodiments, the interference fit connectors 156 and 150 may be reversed. For example, the plunger arm 110 may include one or more I-shaped extrusions configured to be inserted within one or more I-shaped sockets of the plunger rod 108. In some embodiments, the base 106 can also be formed at the proximal end 118 the plunger arm 110 instead of being formed at the proximal end 112 of the plunger rod 108. It is also contemplated that the base 106 may be formed from two base components that join together (e.g., a portion of the base 106 on the plunger rod 108 and a portion of the base 106 on the plunger arm 110). Furthermore, the interference fit connectors 150 and 156 may include different geometries in some embodiments (e.g., X-shaped extrusions/sockets, T-shaped extrusions/sockets, H-shaped extrusions/sockets, or any other cooperatively shaped extrusions/sockets).

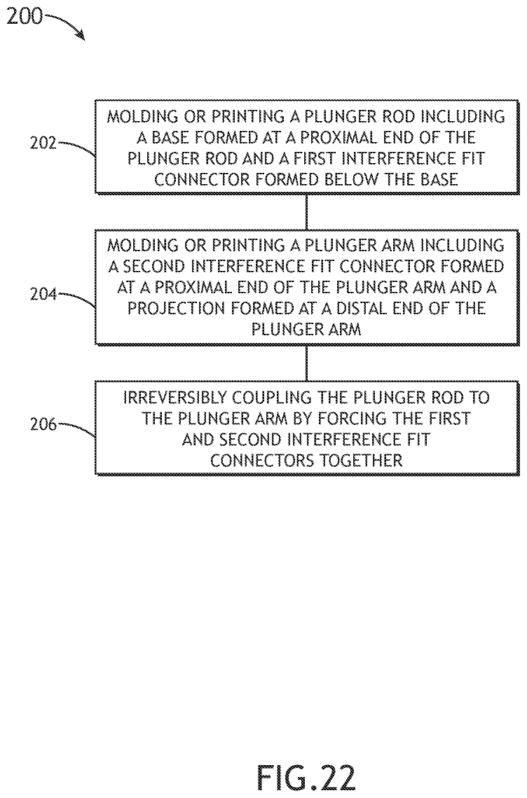

Referring now to FIG. 22, a method 200 of manufacturing the syringe plunger 104 is described. At step 202, the method 200 includes molding (e.g., injection molding) or printing (e.g., 3D printing) the plunger rod 108 including the base 106 formed at the proximal end 112 of the plunger rod 108 and the interference fit connector 156 formed below the base 106 (i.e., the plunger rod 108 as described with reference to FIGS. 12 through 21). At step 204, the method 200 includes molding or printing the plunger arm 110 including the interference fit connector 150 formed at the proximal end 118 of the plunger arm 110 and the projection 122 formed at the distal end 120 of the plunger arm 110 (i.e., the plunger arm 110 as described with reference to FIGS. 12 through 21). At step 206, the method 200 includes irreversibly coupling the plunger rod 108 to the plunger arm 110 by forcing the interference fit connectors 150 and 156 together. In some embodiments of the method 200, the interference fit connectors 150 and 156 are further irreversibly coupled by applying heat or radiation after the interference fit connectors 156 and 150 are forced together. For example, heat or radiation may be used to fuse the interference fit connectors 150 and 156 together. Alternatively, or additionally, the interference fit connectors 150 and 156 may be chemically fused together, or the interference fit connectors 150 and 156 may be bonded together by an adhesive disposed between the interference fit connectors 150 and 156 before the interference fit connectors 150 and 156 are forced together.

The use of separately manufactured plunger rod 108 and plunger arm 110 components may be particularly advantageous in embodiments where the components are manufactured in bulk by injection molding. By manufacturing the plunger rod 108 and the plunger arm 110 as separate components, the syringe plunger 104 can be produced from smaller and simpler shapes. Consequently, the manufacturing process may be faster, employ a more efficient use of material, and/or result in a syringe plunger with higher structural integrity.

As used herein, “irreversible” coupling does not necessarily mean that components (e.g., the plunger rod 108 and plunger arm 110) cannot be separated at all. Those skilled in the art will appreciate that any components that are fastened or adhered together may be separated through the use of sufficient force or specialized tools/processes. Thus, the term “irreversible” coupling is used herein to indicated that the components cannot be separated without the use of specialized tools/processes or excessive force that may damage one or more of the components.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

What is claimed is:

1. A syringe plunger, comprising:
a plunger rod including a base formed at a proximal end of the plunger rod, the plunger rod further including a first interference fit connector formed below the base at the proximal end of the plunger rod, the plunger rod configured to extend longitudinally from the base into a syringe barrel; and
a plunger arm including a second interference fit connector formed at a proximal end of the plunger arm, the second interference fit connector configured to be coupled to the first interference fit connector so that the plunger arm extends longitudinally from the base along an outer surface of the syringe barrel, the plunger arm further including a projection formed at a distal end of the plunger arm, the projection configured to receive an applied force that causes an entirety of the syringe plunger to move relative to the syringe barrel so that the plunger rod is actuated through a portion of the syringe barrel,
wherein the plunger rod and the plunger arm are separately manufactured and irreversibly coupled by forcing the first and second interference fit connectors together.

2. The syringe plunger of claim 1, wherein the plunger rod and the plunger arm are separately manufactured by injection molding.

3. The syringe plunger of claim 1, wherein the first interference fit connector of the plunger rod comprises one or more I-shaped extrusions.

4. The syringe plunger of claim 1, wherein the second interference fit connector of the plunger arm comprises one or more I-shaped sockets.

5. The syringe plunger of claim 1, wherein the second interference fit connector is partially curved to conform to a shape of the plunger rod.

6. The syringe plunger of claim 1, wherein the first interference fit connector is configured to be disposed within the second interference fit connector to form an irreversible coupling directly below the base of the plunger rod, such that the base of the plunger rod and the irreversible coupling form a base structure for the entirety of the syringe plunger.

7. The syringe plunger of claim 1, wherein the first and second interference fit connectors are further irreversibly coupled by an application of heat or radiation after the first and second interference fit connectors are forced together.

8. The syringe plunger of claim 1, wherein the first and second interference fit connectors are further irreversibly coupled by an adhesive disposed between the first and second interference fit connectors before the first and second interference fit connectors are forced together.

9. The syringe plunger of claim 1, wherein the projection comprises a circular sector or segment with an arc measure in the range of 90 to 180 degrees, the circular sector or segment configured to be pushed by a finger of a user to cause the entirety of the syringe plunger to move relative to the syringe barrel.

10. The syringe plunger of claim 9, wherein the plunger arm includes an indented portion above the circular sector or segment, the indented portion of the plunger arm having a curvature configured to conform to the finger of the user for improved grip.

11. The syringe plunger of claim 10, wherein the indented portion of the plunger arm further includes surface texturing for improved grip.

12. The syringe plunger of claim 1, wherein a lower portion of the plunger arm is configured to contact the syringe barrel, and an upper portion of the plunger arm is outdented from the lower portion of the plunger arm to prevent or reduce contact between the upper portion of the plunger arm and the syringe barrel for reduced friction between the plunger arm and the syringe barrel when the entirety of the syringe plunger to moves relative to the syringe barrel.

13. The syringe plunger of claim 12, wherein the lower portion of the plunger arm further includes a smooth bump configured to contact the syringe barrel and configured to prevent or reduce contact between a remainder of the lower portion of the plunger arm and the syringe barrel for reduced friction between the plunger arm and the syringe barrel when the entirety of the syringe plunger to moves relative to the syringe barrel.

14. A method of manufacturing a syringe plunger, comprising:
molding or printing a plunger rod including a base formed at a proximal end of the plunger rod, the plunger rod further including a first interference fit connector formed below the base at the proximal end of the plunger rod, the plunger rod configured to extend longitudinally from the base into a syringe barrel;
molding or printing a plunger arm including a second interference fit connector formed at a proximal end of the plunger arm, the second interference fit connector configured to be coupled to the first interference fit connector so that the plunger arm extends longitudinally from the base along an outer surface of the syringe barrel, the plunger arm further including a projection formed at a distal end of the plunger arm, the projection configured to receive an applied force that causes an entirety of the syringe plunger to move relative to the syringe barrel so that the plunger rod is actuated through a portion of the syringe barrel; and
irreversibly coupling the plunger rod to the plunger arm by forcing the first and second interference fit connectors together.

15. The method of claim 14, wherein the plunger rod and the plunger arm are separately manufactured by injection molding.

16. The method of claim 14, wherein the first interference fit connector is disposed within the second interference fit connector to form an irreversible coupling directly below the base of the plunger rod, such that the base of the plunger rod and the irreversible coupling form a base structure for the entirety of the syringe plunger.

17. The method of claim 14, wherein the first and second interference fit connectors are further irreversibly coupled by applying heat or radiation after the first and second interference fit connectors are forced together.

18. The method of claim 14, wherein the first and second interference fit connectors are further irreversibly coupled by disposing an adhesive between the first and second interference fit connectors before the first and second interference fit connectors are forced together.

19. The method of claim 14, wherein a lower portion of the plunger arm is configured to contact the syringe barrel, and an upper portion of the plunger arm is outdented from the lower portion of the plunger arm to prevent or reduce contact between the upper portion of the plunger arm and the syringe barrel for reduced friction between the plunger arm and the syringe barrel when the entirety of the syringe plunger to moves relative to the syringe barrel.

20. The method of claim 19, wherein the lower portion of the plunger arm further includes a smooth bump configured to contact the syringe barrel and configured to prevent or reduce contact between a remainder of the lower portion of the plunger arm and the syringe barrel for reduced friction between the plunger arm and the syringe barrel when the entirety of the syringe plunger to moves relative to the syringe barrel.
